(19) United States
(12) Patent Application Publication
(10) Pub. No.: US 2017/0260492 A1
(43) Pub. Date: Sep. 14, 2017

(54) TEMPERATURE STABILIZED CULTURE INCUBATOR

(71) Applicant: Tokitae LLC, Bellevue, WA (US)

(72) Inventors: RYAN CALDERON, SEATTLE, WA (US); MICHAEL FRIEND, SEATTLE, WA (US); SIMON JEFFREY GHIONEA, NORTH BEND, WA (US); ANDREW KUEHL MILLER, SEATTLE, WA (US); BRYAN NORTON, SEATTLE, WA (US)

(21) Appl. No.: 15/063,833

(22) Filed: Mar. 8, 2016

(51) Int. Cl. C12M 1/00; G01N 29/00; C12Q 3/00; G01N 25/02; C12M 1/36; C12M 1/34

(52) U.S. Cl. CPC C12M 41/14; C12M 41/48; C12M 23/38; C12M 41/44; C12Q 3/00; G01N 25/02; G01N 29/00; G01N 2291/025

(57) ABSTRACT

Described embodiments include a culture incubator, method, and sensor circuit. A culture incubator includes an accessible incubation compartment configured to contain a culture item at a specified incubation temperature; a phase change material having a phase transition temperature over the specified incubation temperature; and a heat transfer element in thermal communication with the phase change material and configured to transfer heat to the phase change material. A sensor circuit is configured to acquire data indicative of a phase composition state of the phase change material. A manager circuit is configured to determine a difference between the phase composition state and a target phase composition state for the phase change material. A controller circuit is configured to transfer heat to the phase change material in an amount estimated to change the phase composition state of the phase change material to the target phase composition state.

If an Application Data Sheet (ADS) has been filed on the filing date of this application, it is incorporated by reference herein. Any applications claimed on the ADS for priority under 35 U.S.C. §§119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application claims the benefit of the earliest available effective filing date(s) from the following listed application(s) (the “Priority Applications”), if any, listed below (e.g., claims earliest available priority dates for other than provisional patent applications or claims benefits under 35 USC §119(e) for provisional patent applications, for any and all parent, grandparent, great-grandparent, etc. applications of the Priority Application(s)). In addition, the present application is related to the “Related Applications,” if any, listed below.

### Priority Applications

None.

If the listings of applications provided above are inconsistent with the listings provided via an ADS, it is the intent of the Applicant to claim priority to each application that appears in the Priority Applications section of the ADS and to each application that appears in the Priority Applications section of this application.

All subject matter of the Priority Applications and the Related Applications and of any and all parent, grandparent, great-grandparent, etc. applications of the Priority Applications and the Related Applications, including any priority claims, is incorporated herein by reference to the extent such subject matter is not inconsistent herewith.

### SUMMARY

For example, and without limitation, an embodiment of the subject matter described herein includes a culture incubator. The culture incubator includes an accessible incubation compartment configured to contain a culture item at a specified incubation temperature. The culture incubator includes a phase change material having a phase transition temperature over the specified incubation temperature and in thermal communication with the incubation compartment. The culture incubator includes a heat transfer element in thermal communication with the phase change material and configured to transfer heat to the phase change material. The culture incubator includes a sensor circuit configured to acquire data indicative of a current phase composition state of the phase change material. The culture incubator includes a PCM manager circuit configured to determine in response to the data indicative of a current phase composition state a difference between the current phase composition state and a target phase composition state for the phase change material. The culture incubator includes a controller circuit configured to transfer heat from the heat transfer element to the phase change material in an amount estimated to change the current phase composition state of the phase change material to the target phase composition state.

In an embodiment, the culture incubator includes thermal insulation configured to thermally separate the phase change material and the incubation compartment from an ambient environment.

For example, and without limitation, an embodiment of the subject matter described herein includes a method for maintaining a specified incubation temperature in an accessible incubation compartment of a culture incubator. The method includes acquiring data indicative of a phase composition state of a phase change material in thermal communication with the accessible incubation compartment. The method includes determining in response to the data indicative of a phase composition state a difference between the phase composition state and a target phase composition state. The method includes transferring heat (+Q or −Q) from a heat transfer element to the phase change material in an amount estimated to change the phase composition state of the phase change material to the target phase composition state.

For example, and without limitation, an embodiment of the subject matter described herein includes a sensor circuit for determining a phase composition state of a phase change material. The sensor circuit includes an ultrasound transmitter configured to emit ultrasound waves into the phase change material. The sensor circuit includes an ultrasound receiver configured to receive the ultrasound waves directed into the phase change material by the ultrasound transmitter. The sensor circuit includes circuitry for measuring time of flight over a known distance through the phase change material by ultrasound waves emitted by the ultrasound transmitter and received by the ultrasound receiver. The sensor circuit includes circuitry for correlating the time of flight with a particular phase composition state of the phase change material.

In an embodiment, the sensor circuit includes an ultrasound transducer that includes the ultrasound transmitter and the ultrasound receiver, and an ultrasound reflector.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 illustrates an example embodiment of an environment 100 in which embodiments may be implemented;

FIG. 2 illustrates an example operational flow 200 for maintaining a specified incubation temperature in an accessible incubation compartment of a culture incubator; and

FIG. 3 illustrates an example environment 300 in which embodiments may be implemented.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Those having skill in the art will recognize that the state of the art has progressed to the point where there is little distinction left between hardware, software, and/or firmware implementations of aspects of systems; the use of hardware, software, and/or firmware is generally (but not always, in that in certain contexts the choice between hardware and software can become significant) a design choice representing cost vs. efficiency tradeoffs. Those having skill in the art will appreciate that there are various implementations by which processes and/or systems and/or other technologies described herein can be effected (e.g., hardware, software, and/or firmware), and that the preferred implementation will vary with the context in which the processes and/or systems and/or other technologies are deployed. For example, if an implementer determines that speed and accuracy are paramount, the implementer may opt for a mainly hardware and/or firmware implementation; alternatively, if flexibility is paramount, the implementer may opt for a mainly software implementation; or, yet again alternatively, the implementer may opt for some combination of hardware, software, and/or firmware. Hence, there are several possible implementations by which the processes and/or devices and/or other technologies described herein may be effected, none of which is inherently superior to the other in that any implementation to be utilized is a choice dependent upon the context in which the implementation will be deployed and the specific concerns (e.g., speed, flexibility, or predictability) of the implementer, any of which may vary. Those skilled in the art will recognize that optical aspects of implementations will typically employ optically-oriented hardware, software, and or firmware.

In some implementations described herein, logic and similar implementations may include software or other control structures suitable to implement an operation. Electronic circuitry, for example, may manifest one or more paths of electrical current constructed and arranged to implement various logic functions as described herein. In some implementations, one or more media are configured to bear a device-detectable implementation if such media hold or transmit a special-purpose device instruction set operable to perform as described herein. In some variants, for example, this may manifest as an update or other modification of existing software or firmware, or of gate arrays or other programmable hardware, such as by performing a reception of or a transmission of one or more instructions in relation to one or more operations described herein. Alternatively or additionally, in some variants, an implementation may include special-purpose hardware, software, firmware components, and/or general-purpose components executing or otherwise invoking special-purpose components. Specifications or other implementations may be transmitted by one or more instances of tangible transmission media as described herein, optionally by packet transmission or otherwise by passing through distributed media at various times.

Alternatively or additionally, implementations may include executing a special-purpose instruction sequence or otherwise invoking circuitry for enabling, triggering, coordinating, requesting, or otherwise causing one or more occurrences of any functional operations described below. In some variants, operational or other logical descriptions herein may be expressed directly as source code and compiled or otherwise invoked as an executable instruction sequence. In some contexts, for example, C++ or other code sequences can be compiled directly or otherwise implemented in high-level descriptor languages (e.g., a logic-synthesizable language, a hardware description language, a hardware design simulation, and/or other such similar mode(s) of expression). Alternatively or additionally, some or all of the logical expression may be manifested as a Verilog-type hardware description or other circuitry model before physical implementation in hardware, especially for basic operations or timing-critical applications. Those skilled in the art will recognize how to obtain, configure, and optimize suitable transmission or computational elements, material supplies, actuators, or other common structures in light of these teachings.

In a general sense, those skilled in the art will recognize that the various embodiments described herein can be implemented, individually and/or collectively, by various types of electro-mechanical systems having a wide range of electrical components such as hardware, software, firmware, and/or virtually any combination thereof; and a wide range of components that may impart mechanical force or motion such as rigid bodies, spring or torsional bodies, hydraulics, electro-magnetically actuated devices, and/or virtually any combination thereof. Consequently, as used herein “electro-mechanical system” includes, but is not limited to, electrical circuitry operably coupled with a transducer (e.g., an actuator, a motor, a piezoelectric crystal, a Micro Electro Mechanical System (MEMS), etc.), electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), electrical circuitry forming a communications device (e.g., a modem, module, communications switch, optical-electrical equipment, etc.), and/or any non-electrical analog thereto, such as optical or other analogs. Those skilled in the art will also appreciate that examples of electro-mechanical systems include but are not limited to a variety of consumer electronics systems, medical devices, as well as other systems such as motorized transport systems, factory automation systems, security systems, and/or communication/computing systems. Those skilled in the art will recognize that electro-mechanical as used herein is not necessarily limited to a system that has both electrical and mechanical actuation except as context may dictate otherwise.

In a general sense, those skilled in the art will also recognize that the various aspects described herein which can be implemented, individually and/or collectively, by a wide range of hardware, software, firmware, and/or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), and/or electrical circuitry forming a communications device (e.g., a modem, communications switch, optical-electrical equipment, etc.). Those having skill in the art will recognize that the subject matter described herein may be implemented in an analog or digital fashion or some combination thereof.

Those skilled in the art will further recognize that at least a portion of the devices and/or processes described herein can be integrated into an image processing system. A typical image processing system may generally include one or more of a system unit housing, a video display device, memory such as volatile or non-volatile memory, processors such as microprocessors or digital signal processors, computational entities such as operating systems, drivers, applications programs, one or more interaction devices (e.g., a touch pad, a touch-sensitive screen or display surface, an antenna, etc.), control systems including feedback loops and control motors (e.g., feedback for sensing lens position and/or velocity; control motors for moving/distorting lenses to give desired focuses). An image processing system may be implemented utilizing suitable commercially available components, such as those typically found in digital still systems and/or digital motion systems.

Those skilled in the art will likewise recognize that at least some of the devices and/or processes described herein can be integrated into a data processing system. Those having skill in the art will recognize that a data processing system generally includes one or more of a system unit housing, a video display device, memory such as volatile or non-volatile memory, processors such as microprocessors or digital signal processors, computational entities such as operating systems, drivers, graphical user interfaces, and applications programs, one or more interaction devices (e.g., a touch pad, a touch-sensitive screen or display surface, an antenna, etc.), and/or control systems including feedback loops and control motors (e.g., feedback for sensing position and/or velocity; control motors for moving and/or adjusting components and/or quantities). A data processing system may be implemented utilizing suitable commercially available components, such as those typically found in data computing/communication and/or network computing/communication systems.

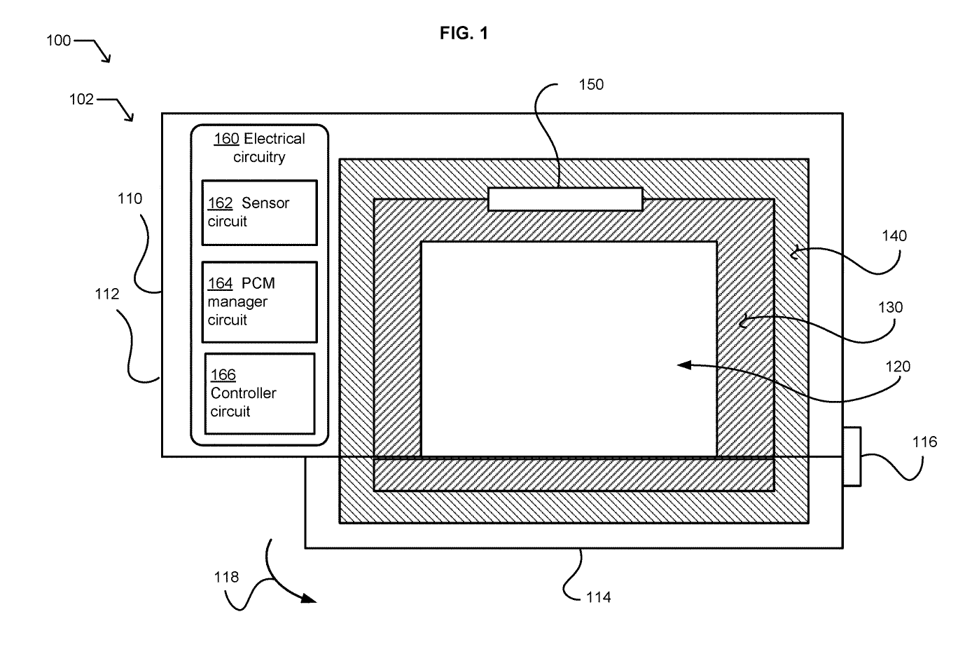

FIG. 1 illustrates an example environment 100 in which embodiments may be implemented. The environment includes a culture incubator 110 in an ambient environment 102 surrounding the culture incubator. The culture incubator includes an accessible incubation compartment 120 configured to contain a culture item at a specified incubation temperature. For example, the culture item may include a tuberculous specimen. The culture incubator includes a body 112 and a hinged 116 door 114. The incubation compartment may be accessed by opening 118 the door 114 of the culture incubator. The culture incubator includes a phase change material 130 having a phase transition temperature over the specified incubation temperature and in thermal communication with the incubation compartment. In an embodiment, the phase transition temperature includes a solid-liquid phase transition temperature. In an embodiment, the phase change material is selected to constrain increases or decreases in the temperature in the incubation compartment during a power outage event. In an embodiment, the phase-change material includes a phase change composite material. In an embodiment, the phase-change material is a substance with a high heat of fusion which, melting and solidifying at a certain temperature, is capable of storing and releasing large amounts of energy. In an embodiment, the phase-change material is a material that changes its state between solid and liquid or between two different solid crystallization states over a defined temperature range (i.e. phase transition). This process is reversible (i.e., a reproducible phase transition).

The culture incubator 110 includes a heat transfer element 150 in thermal communication with the phase change material 130 and configured to transfer heat to the phase change material. For example, the heat transfer may be positive (+Q) or negative (−Q). The culture incubator includes electrical circuitry 160. The electrical circuitry includes a sensor circuit 162 configured to acquire data indicative of a current phase composition state of the phase change material. For example, a current phase composition state may include a mixed composition of a solid state and a liquid state of the phase change material. For example, a current phase composition state may include an all solid state of the phase change material, or an all liquid state of the phase change material. For example, a current phase composition state may include a freeze/melt ratio of the phase change material. For example, a current phase composition state of the phase change material may include how much mass is solid and how much mass is liquid, or much volume is solid and how much volume is liquid. The electrical circuitry includes a PCM manager circuit 164 configured to determine in response to the data indicative of a phase composition state a difference between the phase composition state and a target phase composition state for the phase change material. The electrical circuitry includes a controller circuit 166 configured to transfer heat from the heat transfer element 150 to the phase change material in an amount estimated to change the phase composition state of the phase change material to the target phase composition state. For example, the controller will transfer positive heat (+Q) if too much of the phase change material is solid. For example, in this situation the controller heats the heat transfer element to a temperate over a period of time estimated to change the phase composition state of the phase change material to the target phase composition state. For example, the controller will transfer negative heat (−Q) if too much of the phase change material is liquid. For example, in this situation the controller cools the heat transfer element to a temperate over a period of time estimated to change the phase composition state of the phase change material to the target phase composition state. In an embodiment, the controller circuit is configured to transfer heat from the heat transfer element to the phase change material in an amount estimated to change the phase composition state of the phase change material to the target phase composition state while maintaining the specified incubation temperature.

In an embodiment, the accessible incubation compartment 120 includes an accessible incubation compartment configured to contain a culture item at a specified incubation temperature and to allow insertion and removal of the culture item. In an embodiment, the accessible incubation compartment includes a door or hatch providing access to the incubation compartment. In an embodiment, the specified incubation temperature is selected from a temperature between approximately 30° C. and approximately 37° C. In an embodiment, the specified incubation temperature is selected as 36° C. with a tolerated range of approximately +/−1° C. In an embodiment, the specified incubation temperature is selected as 37° C. with a tolerated range of approximately +/−1° C.

In an embodiment, the phase change material 130 includes a paraffin wax having a phase transition temperature over the specified incubation temperature. In an embodiment, the phase change material includes a mixture of paraffin waxes having at least two different chain lengths and providing a continuous phase transition over a temperature range over the specified incubation temperature. In an embodiment, the phase change material includes hydrated salts having a phase transition temperature over the specified incubation temperature. In an embodiment, a solid-liquid transition temperature of the phase change material straddles the specified incubation temperature. In an embodiment, a solid-liquid transition temperature of the phase change material includes the specified incubation temperature. In an embodiment, the phase change material includes a sufficient amount of phase change material to maintain the incubation compartment at the specified incubation temperature for at least 12 hours if an ambient temperature of the environment 102 of the culture incubator is within plus or minus 20 degrees Celsius of the specified incubation temperature. In an embodiment, the phase change material includes a sufficient amount of phase change material to maintain the incubation compartment at the specified incubation temperature for at least 24 hours if an ambient temperature of the environment of the culture incubator is within plus or minus 20 degrees Celsius of the specified incubation temperature.

In an embodiment, the phase change material includes a sufficient amount of phase change material to maintain the incubation compartment at the specified incubation temperature if an ambient temperature of the environment 102 of the culture incubator 110 is between 5° C. and 43° C. In an embodiment, the phase change material surrounds at least fifty-percent of an exterior surface of the incubation compartment 120. In an embodiment, the culture incubator includes a heat spreader configured to transfer heat from the heat transfer element 150 to the phase change material.

In an embodiment, the heat transfer element 150 includes a heater configured to transfer heat to the phase change material 130. In an embodiment, the heater includes an electrically resistive heater. In an embodiment, the heat transfer element includes a cooler configured to negatively transfer heat to the phase change material. In an embodiment, the heater includes a thermal electric cooler.

In an embodiment, the sensor circuit 162 includes an ultrasound sensor circuit configured to acquire time of flight data indicative of a phase composition state of the phase change material 130. In an embodiment, the time of flight data is responsive to an average mass temperature of phase change material. For example, ultrasonic pulses may be sent through the phase change material over a distance “d” to a receiver, and a time of flight measured. The time of flight and the density of the phase change material change as the temperature of the phase change material changes. In an embodiment, the ultrasound sensor circuit configured to acquire the time of flight data responsive to the phase composition state of the phase change material. In an embodiment, the sensor circuit includes a volumetric-change sensor circuit configured to acquire volumetric change data indicative of a phase composition state of the phase change material. In an embodiment, the sensor circuit includes an electrical conductivity sensor circuit configured to acquire electrical conductivity data indicative of a phase composition state of the phase change material. In an embodiment, the sensor circuit includes a capacitive-based sensor circuit configured to acquire permittivity data indicative of a phase composition state of the phase change material. For example, in an embodiment, the capacitive-based sensor circuit configured to use a capacitor to measure an imaginary or loss component of the permittivity of the phase change material. In an embodiment, the sensor circuit includes an optical sensor circuit configured to acquire light transmission data indicative of a phase composition state of the phase change material. For example, in an embodiment, light transmitted through a phase change material at wavelengths of 400-1100 nm, 1300 nm, and 1600 nm attenuates in a manner responsive to a phase composition state of the phase change material. In an embodiment, the sensor circuit includes a temperature probe configured to acquire temperature data indicative of a phase composition state of the phase change material. In an embodiment, the sensor circuit includes an array of temperature probes configured to acquire temperature data indicative of a phase composition state of the phase change material. In an embodiment, the target phase composition state is approximately equal parts solid and liquid. In this embodiment, the target phase composition state is approximately equal parts solid and liquid measured by mass or by volume.

In an embodiment, the PCM manager circuit 164 is further configured to determine the target phase composition state in response to an ambient temperature of the environment 102 surrounding the culture incubator 110. In an embodiment, the ambient temperature of the environment surrounding the culture incubator 102 includes a present ambient temperature. In an embodiment, the ambient temperature of the environment surrounding the culture incubator includes a historical ambient temperature. For example, the historical ambient temperature may include a recent 24, 36, or 48-hour average ambient temperature. For example, the historical ambient temperature for a current date or a current month averaged over at least two years. In an embodiment, the ambient temperature of the environment surrounding the culture incubator includes a forecasted ambient temperature. In an embodiment, the PCM manager circuit is further configured to determine the target phase composition state in response to a forecasted event in the environment surrounding the culture incubator. For example, a forecasted event may include a forecasted humidity, a forecasted storm, or a forecasted event correlating to loss of electrical power to the culture incubator. For example, a forecasted event may include a hurricane, riots, or rolling blackouts.

In an embodiment, the controller circuit 166 includes a controller circuit configured to control a positive transfer of heat (+Q) from the heat transfer element 150 to the phase change material 130 in an amount estimated to change the phase composition state of the phase change material to the target phase composition state. In an embodiment, the controller circuit is configured to control a positive transfer of heat (+Q) from the heat transfer element to the phase change material in an amount estimated to change the phase composition state of the phase change material to the target phase composition state while maintaining the specified incubation temperature. In an embodiment, the controller circuit includes a controller circuit configured to control a negative transfer of heat (−Q) from the heat transfer element to the phase change material in an amount estimated to change the phase composition state of the phase change material to the target phase composition state. In an embodiment, the controller circuit includes a controller circuit configured to control a negative transfer of heat (−Q) from the heat transfer element to the phase change material in an amount estimated to change the phase composition state of the phase change material to the target phase composition state while maintaining the specified incubation temperature. In an embodiment, the controller circuit includes a controller circuit configured to transfer heat (+Q or −Q) from the heat transfer element to the phase change material in an amount estimated to change the phase composition state of the phase change material to the target phase composition state while maintaining the specified incubation temperature.

In an embodiment, the culture incubator 110 includes a thermal insulation 140 configured to thermally separate the phase change material 130 and the incubation compartment 120 from the ambient environment 102 surrounding the culture incubator. In an embodiment, the thermal insulation includes a thermal barrier.

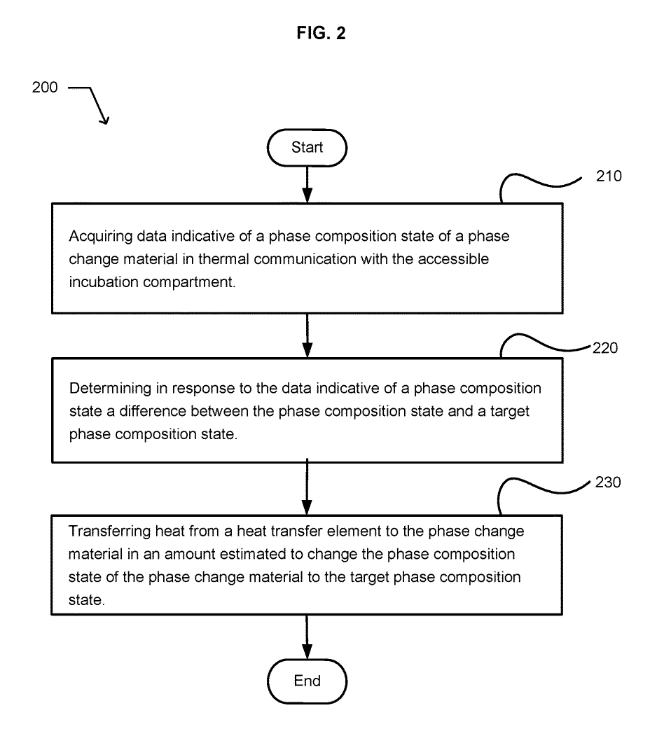

FIG. 2 illustrates an example operational flow 200 for maintaining a specified incubation temperature in an accessible incubation compartment of a culture incubator. FIG. 1 illustrates an example culture incubator 110. After a start operation, the operational flow includes a collection operation 210. The collection operation includes acquiring data indicative of a phase composition state of a phase change material in thermal communication with the accessible incubation compartment. In an embodiment, the collection operation may be implemented using the sensor circuit 162 described in conjunction with FIG. 1. A divergence operation 120 includes determining in response to the data indicative of a phase composition state a difference between the phase composition state and a target phase composition state. In an embodiment, the divergence operation 220 may be implemented using the PCM manager circuit 164 described in conjunction with FIG. 1. A heat-transfer operation 230 includes transferring heat (+Q or −Q) from a heat transfer element to the phase change material in an amount estimated to change the phase composition state of the phase change material to the target phase composition state. In an embodiment, the heat-transfer operation may be implemented using the controller circuit 166 to transfer heat from the heat transfer element 150 to the incubation compartment 120. In an embodiment, the heat-transfer operation includes transferring heat (+Q or −Q) from a heat transfer element to the phase change material in an amount estimated to change the phase composition state of the phase change material to the target phase composition state while maintaining the specified incubation temperature in the incubation compartment.

In an embodiment of the collection operation 210, the acquiring data includes ultrasonically acquiring time of flight data indicative of a phase composition state of the phase change material. In an embodiment, the acquiring data includes acquiring volumetric change data indicative of a phase composition state of the phase change material. In an embodiment, the acquiring data includes acquiring electrical conductivity data indicative of a phase composition state of the phase change material. In an embodiment, the acquiring data includes acquiring permittivity data indicative of a phase composition state of the phase change material. In an embodiment, the acquiring data includes acquiring light transmission data indicative of a phase composition state of the phase change material. In an embodiment, the acquiring data includes acquiring temperature data indicative of a phase composition state of the phase change material.

In an embodiment of the divergence operation 220, the determining includes determining the target phase composition state in response to an ambient temperature of an environment surrounding the culture incubator. In an embodiment, the determining includes determining the target phase composition state in response to a forecasted event in the environment surrounding the culture incubator.

In an embodiment of the heat-transfer operation 220, the transferring heat includes transferring heat from the heat transfer element to the phase change material in an amount estimated to change the phase composition state of the phase change material to the target phase composition state while maintaining the specified incubation temperature. In an embodiment, the transferring heat includes a positive transferring of heat from the heat transfer element to the phase change material in an amount estimated to change the phase composition state of the phase change material to the target phase composition state. In an embodiment, the transferring heat includes a negative transferring of heat from the heat transfer element to the phase change material in an amount estimated to change the phase composition state of the phase change material to the target phase composition state.

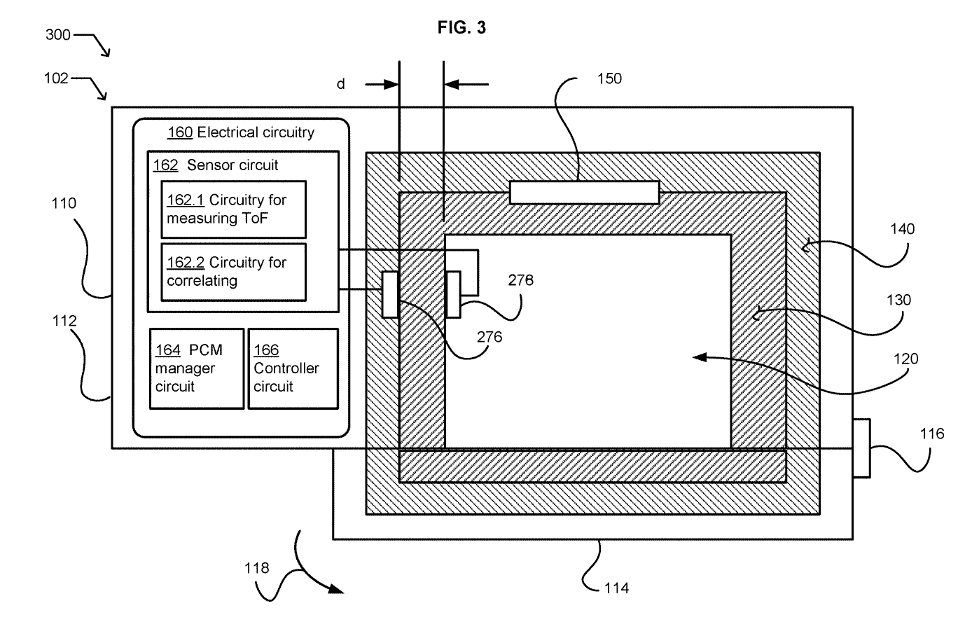

FIG. 3 illustrates an example environment 300 in which embodiments may be implemented. The example environment includes an alternative embodiment of the culture incubator 110. The alternative embodiment of the culture incubator includes an alternative embodiment of the sensor circuit 162 for determining a phase composition state of the phase change material 130. The alternative embodiment of the sensor circuit includes an ultrasound transmitter 276 configured to emit ultrasound waves into the phase change material 130. In an embodiment, the ultrasound transmitter is configured to emit pulsed ultrasound waves into the phase change material. The alternative embodiment of the sensor circuit includes an ultrasound receiver 278 configured to receive the ultrasound waves directed into the phase change material by the ultrasound transmitter. The alternative embodiment of the sensor circuit includes circuitry for measuring time of flight 162.1 over a known distance, illustrated as a known distance d, through the phase change material 130 by ultrasound waves emitted by the ultrasound transmitter and received by the ultrasound receiver. The alternative embodiment of the sensor circuit includes circuitry for correlating 162.2 the time of flight with a particular phase composition state of the phase change material. For example, in an embodiment, a fluid speed of sound in the phase change material may be determined based on the formula

ToF=(known distance d)/(fluid speed of sound in the phase change material)

The fluid speed of sound in the phase change material is correlated with a phase and a temperature of the phase change material. In an alternative embodiment, the sensor circuit 162 includes an ultrasound transducer that includes the ultrasound transmitter 276 and the ultrasound receiver 278 in a single housing. In this alternative embodiment, the sensor circuit includes an ultrasound reflector located at the measured distance d from the ultrasound transducer.

An aspect of the culture incubator 110 is the ability to maintain the phase composition state of the phase change material at a point halfway between melting and freezing. This enables the culture incubator to withstand temperature excursions either above or below the desired set point in power outage conditions. As the phase change occurs under near isothermal conditions it is very difficult to measure the freeze thaw percentage as a function of temperature. The culture incubator instead utilizes a time of flight measurement to determine the phase composition state of the phase change material. Because the speed of sound in a media is a function of the density of the media, which in turn is a function of the phase, the time of flight of a ultrasound pulse provides a good measurement of the phase composition state of the phase change material in the path over a known distance. This is achieved using ultrasonic pulses which are sent from the transmitter 276 to a receiver 278 over the distance d with the transit time being measured. In an embodiment, the ultrasound transmitter 276 and the ultrasound receiver receivers are attached to a structure containing the phase change material. As the phase change material freezes, its temperature drops below its freezing point , its density increases, the speed of sound increases, and the time of flight of the ultrasonic pulse decreases.

All references cited herein are hereby incorporated by reference in their entirety or to the extent their subject matter is not otherwise inconsistent herewith.

In some embodiments, “configured” or “ configured to” includes at least one of designed, set up, shaped, implemented, constructed, or adapted for at least one of a particular purpose, application, or function. In some embodiments, “configured” or “configured to” includes positioned, oriented, or structured for at least one of a particular purpose, application, or function.

It will be understood that, in general, terms used herein, and especially in the appended claims, are generally intended as “open” terms. For example, the term “including” should be interpreted as “including but not limited to.” For example, the term “having” should be interpreted as “having at least.” For example, the term “has” should be interpreted as “having at least.” For example, the term “includes” should be interpreted as “includes but is not limited to,” etc. It will be further understood that if a specific number of an introduced claim recitation is intended, such an intent will be explicitly recited in the claim, and in the absence of such recitation no such intent is present. For example, as an aid to understanding, the following appended claims may contain usage of introductory phrases such as “at least one” or “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a receiver” should typically be interpreted to mean “at least one receiver”); the same holds true for the use of definite articles used to introduce claim recitations. In addition, even if a specific number of an introduced claim recitation is explicitly recited, it will be recognized that such recitation should typically be interpreted to mean at least the recited number (e.g., the bare recitation of “at least two chambers,” or “a plurality of chambers,” without other modifiers, typically means at least two chambers).

In those instances where a phrase such as “at least one of A, B, and C,” “at least one of A, B, or C,” or “an [item] selected from the group consisting of A, B, and C,” is used, in general such a construction is intended to be disjunctive (e.g., any of these phrases would include but not be limited to systems that have A alone, B alone, C alone, A and B together, A and C together, B and C together, or A, B, and C together, and may further include more than one of A, B, or C, such as A₁, A₂, and C together, A, B₁, B₂, C₁, and C₂ together, or B₁ and B₂ together). It will be further understood that virtually any disjunctive word or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

The herein described aspects depict different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality. Any two components capable of being so associated can also be viewed as being “operably couplable” to each other to achieve the desired functionality. Specific examples of operably couplable include but are not limited to physically mateable or physically interacting components or wirelessly interactable or wirelessly interacting components.

With respect to the appended claims the recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Use of “Start,” “End,” “Stop,” or the like blocks in the block diagrams is not intended to indicate a limitation on the beginning or end of any operations or functions in the diagram. Such flowcharts or diagrams may be incorporated into other flowcharts or diagrams where additional functions are performed before or after the functions shown in the diagrams of this application. Furthermore, terms like “responsive to,” “related to,” or other past-tense adjectives are generally not intended to exclude such variants, unless context dictates otherwise.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A culture incubator comprising:
an accessible incubation compartment configured to contain a culture item at a specified incubation temperature;
a phase change material having a phase transition temperature over the specified incubation temperature and in thermal communication with the incubation compartment;
a heat transfer element in thermal communication with the phase change material and configured to transfer heat to the phase change material;
a sensor circuit configured to acquire data indicative of a phase composition state of the phase change material;
a PCM manager circuit configured to determine in response to the data indicative of a phase composition state a difference between the phase composition state and a target phase composition state for the phase change material; and
a controller circuit configured to transfer heat from the heat transfer element to the phase change material in an amount estimated to change the phase composition state of the phase change material to the target phase composition state.

2. The culture incubator of claim 1, wherein the accessible incubation compartment includes an accessible incubation compartment configured to contain a culture item at a specified incubation temperature and to allow insertion and removal of the culture item.

3. The culture incubator of claim 1, wherein the accessible incubation compartment includes a door or hatch providing access to the incubation compartment.

4. The culture incubator of claim 1, wherein the specified incubation temperature is selected from a temperature between approximately 30° C. and approximately 37° C.

5. The culture incubator of claim 1, wherein the phase change material includes a paraffin wax having a phase transition temperature over the specified incubation temperature.

6. The culture incubator of claim 1, wherein the phase change material includes a mixture of paraffin waxes having at least two different chain lengths and providing a continuous phase transition over a temperature range over the specified incubation temperature.

7. The culture incubator of claim 1, wherein the phase change material includes hydrated salts having a phase transition temperature over the specified incubation temperature.

8. The culture incubator of claim 1, wherein a solid-liquid transition temperature of the phase change material straddles the specified incubation temperature.

9. The culture incubator of claim 1, wherein a solid-liquid transition temperature of the phase change material includes the specified incubation temperature.

10. The culture incubator of claim 1, wherein the phase change material includes a sufficient amount of phase change material to maintain the incubation compartment at the specified incubation temperature for at least 12 hours if an ambient temperature of an environment of the culture incubator is within plus or minus 20 degrees Celsius of the specified incubation temperature.

11. The culture incubator of claim 1, wherein the phase change material surrounds at least fifty-percent of the exterior surface of the incubation compartment.

12. The culture incubator of claim 1, wherein the heat transfer element includes a heater configured to transfer heat to the phase change material.

13. The culture incubator of claim 1, wherein the heat transfer element includes a cooler configured to negatively transfer heat to the phase change material.

14. The culture incubator of claim 1, wherein the sensor circuit includes an ultrasound sensor circuit configured to acquire time of flight data indicative of a phase composition state of the phase change material.

15. The culture incubator of claim 14, wherein the ultrasound sensor circuit configured to acquire the time of flight data responsive to the phase composition state of the phase change material.

16. The culture incubator of claim 1, wherein the sensor circuit includes a volumetric-change sensor circuit configured to acquire volumetric change data indicative of a phase composition state of the phase change material.

17. The culture incubator of claim 1, wherein the sensor circuit includes an electrical conductivity sensor circuit configured to acquire electrical conductivity data indicative of a phase composition state of the phase change material.

18. The culture incubator of claim 1, wherein the sensor circuit includes a capacitive-based sensor circuit configured to acquire permittivity data indicative of a phase composition state of the phase change material.

19. The culture incubator of claim 1, wherein the sensor circuit includes an optical sensor circuit configured to acquire light transmission data indicative of a phase composition state of the phase change material.

20. The culture incubator of claim 1, wherein the sensor circuit includes a temperature probe configured to acquire temperature data indicative of a phase composition state of the phase change material.

21. The culture incubator of claim 1, wherein the sensor circuit includes an array of temperature probes configured to acquire temperature data indicative of a phase composition state of the phase change material.

22. The culture incubator of claim 1, wherein the target phase composition state is approximately equal parts solid and liquid.

23. The culture incubator of claim 1, wherein the PCM manager circuit is further configured to determine the target phase composition state in response to an ambient temperature of an environment surrounding the culture incubator.

24. The culture incubator of claim 23, wherein the ambient temperature of the environment surrounding the culture incubator includes a present ambient temperature.

25. The culture incubator of claim 23, wherein the ambient temperature of the environment surrounding the culture incubator includes a historical ambient temperature.

26. The culture incubator of claim 23, wherein the ambient temperature of the environment surrounding the culture incubator includes a forecasted ambient temperature.

27. The culture incubator of claim 1, wherein the PCM manager circuit is further configured to determine the target phase composition state in response to a forecasted event in the environment surrounding the culture incubator.

28. The culture incubator of claim 1, wherein the controller circuit includes a controller circuit configured to control a positive transfer of heat (+Q) from the heat transfer element to the phase change material in an amount estimated to change the phase composition state of the phase change material to the target phase composition state.

29. The culture incubator of claim 1, wherein the controller circuit includes a controller circuit configured to control a negative transfer of heat (−Q) from the heat transfer element to the phase change material in an amount estimated to change the phase composition state of the phase change material to the target phase composition state.

30. The culture incubator of claim 1, wherein the controller circuit includes a controller circuit configured to transfer heat (+Q or −Q) from the heat transfer element to the phase change material in an amount estimated to change the phase composition state of the phase change material to the target phase composition state while maintaining the specified incubation temperature.

31. The culture incubator of claim 1, further comprising:
thermal insulation configured to thermally separate the phase change material and the incubation compartment from an ambient environment.

32. A method for maintaining a specified incubation temperature in an accessible incubation compartment of a culture incubator, the method comprising:
acquiring data indicative of a phase composition state of a phase change material in thermal communication with the accessible incubation compartment;
determining in response to the data indicative of a phase composition state a difference between the phase composition state and a target phase composition state; and
transferring heat (+Q or −Q) from a heat transfer element to the phase change material in an amount estimated to change the phase composition state of the phase change material to the target phase composition state.

33. The method of claim 32, wherein the acquiring data includes ultrasonically acquiring time of flight data indicative of a phase composition state of the phase change material.

34. The method of claim 32, wherein the acquiring data includes acquiring volumetric change data indicative of a phase composition state of the phase change material.

35. The method of claim 32, wherein the acquiring data includes acquiring electrical conductivity data indicative of a phase composition state of the phase change material.

36. The method of claim 32, wherein the acquiring data includes acquiring permittivity data indicative of a phase composition state of the phase change material.

37. The method of claim 32, wherein the acquiring data includes acquiring light transmission data indicative of a phase composition state of the phase change material.

38. The method of claim 32, wherein the acquiring data includes acquiring temperature data indicative of a phase composition state of the phase change material.

39. The method of claim 32, wherein the determining includes determining the target phase composition state in response to an ambient temperature of an environment surrounding the culture incubator.

40. The method of claim 32, wherein the determining includes determining the target phase composition state in response to a forecasted event in the environment surrounding the culture incubator.

41. The method of claim 32, wherein the transferring heat includes transferring heat from the heat transfer element to the phase change material in an amount estimated to change the phase composition state of the phase change material to the target phase composition state while maintaining the specified incubation temperature.

42. The method of claim 32, wherein the transferring heat includes a positive transferring of heat from the heat transfer element to the phase change material in an amount estimated to change the phase composition state of the phase change material to the target phase composition state.

43. The method of claim 32, wherein the transferring heat includes a negative transferring of heat from the heat transfer element to the phase change material in an amount estimated to change the phase composition state of the phase change material to the target phase composition state.

44. A sensor circuit for determining a phase composition state of a phase change material, the sensor circuit comprising:
an ultrasound transmitter configured to emit ultrasound waves into the phase change material;
an ultrasound receiver configured to receive the ultrasound waves directed into the phase change material by the ultrasound transmitter;
circuitry for measuring time of flight over a known distance through the phase change material by ultrasound waves emitted by the ultrasound transmitter and received by the ultrasound receiver;
circuitry for correlating the time of flight with a particular phase composition state of the phase change material.

45. The sensor circuit of claim 44, further comprising:
an ultrasound transducer that includes the ultrasound transmitter and the ultrasound receiver; and
an ultrasound reflector.
